(19) United States
(12) Patent Application Publication
(10) Pub. No.: US 2006/0177503 A1
(43) Pub. Date: Aug. 10, 2006

(54) Bambuterol and integrin inhibitor combination and treatment method

(76) Inventors: Alexis Rames, Saint-Louis (FR); Harpreet K. Sandhu, West Orange, NJ (US); David J. Valacer, San Francisco, CA (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/348,386

(22) Filed: Feb. 6, 2006

Related U.S. Application Data
(60) Provisional application No. 60/650,664, filed on Feb. 7, 2005.

(51) Int. Cl. A61K 38/04; A61K 31/325

(52) U.S. Cl. 424/464; 514/19; 514/483

(57) ABSTRACT

The present invention provides novel solid pharmaceutical dosage forms for oral administration comprising a therapeutically active amount of bambuterol, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients. These novel solid pharmaceutical dosage forms are useful in the treatment or control of asthma. The present invention also provides a method for treating asthma employing the solid pharmaceutical dosage forms and a method for preparing the pharmaceutical dosage forms.

### PRIORITY TO RELATED APPLICATIONS

This application claims the benefit of U.S. Provisional Application No. 60/650,664, filed Feb. 7, 2005, which is hereby incorporated by reference in its entirety.

### FIELD OF THE INVENTION

The present invention provides novel solid pharmaceutical dosage forms for oral administration comprising a therapeutically active amount of bambuterol, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients. These novel solid pharmaceutical dosage forms are useful in the treatment or control of asthma and allergic rhinitis. The present invention also provides a method for treating asthma employing the solid pharmaceutical dosage forms and a method for preparing the pharmaceutical dosage forms.

### BACKGROUND OF THE INVENTION

Asthma

Asthma is a chronic inflammatory disorder of the airways characterized by a reduction in lung function and airway hyper-responsiveness (AHR). The airway abnormalities in asthmatics are characterized by constriction, which is the tightening of the smooth muscles surrounding the airways, and inflammation, which is the swelling and irritation of the airways and mucus plugging of small airways caused by mucus hypersecretion. Constriction, plugging and mucosal inflammation contribute to obstruction of airflow, which results in symptoms such as wheezing, coughing, chest tightness, and shortness of breath.

Airway inflammation is a hallmark of asthma. Several studies have documented an association between the numbers of eosinophils and activated lymphocytes in the airways and clinical indices of disease severity. Eosinophils are thought to be important effectors involved in bronchial mucosal damage by the release of cationic proteins, reactive oxygen species, and proinflammatory and profibrotic mediators. Much emphasis has been placed on CD4+ T helper type 2 (Th2) cells as central promulgators of this inflammatory process. These Th2 lymphocytes are believed to orchestrate the events leading to the development of allergic airway responses mainly through the production of Th2-type mediators, which in turn promote the eosinophil-rich infiltrate that distinguishes asthmatic airway inflammation. Although there are available therapies focused on reducing this chronic inflammatory process in asthma, no currently available treatment has been shown to eliminate all features of the disease as a singularly effective treatment. Significant unmet medical needs remain in asthma management for patients with moderate to severe disease.

Early treatment for asthma is focused on relief of the smooth muscle contraction that leads to bronchoconstriction. A variety of medications have been used to provide quick relief and/or prevent bronchoconstriction and the resultant symptoms, e.g., wheeze, cough, exercise intolerance, and/or shortness of breath. Widely used relievers of bronchoconstriction include inhaled short-acting beta-adrenoceptor agonists such as salbutamol and albuterol, their long acting inhaled counterparts, salmeterol and fomoterol and orally administered long acting bambuterol. In addition to these inhaled beta-adrenoceptor agonists, there are controller medications that reduce airway inflammation through daily administration on a long-term basis. Inhaled corticosteroids (ICS) are the most potent and effective anti-inflammatory medications and are the first line of therapy for asthma patients. After a decade of widespread use of inhaled corticosteroids therapy, several respiratory health organizations have produced survey data, which concludes that a majority of moderate to severe asthma patients do not enjoy complete and optimal control of their symptoms as defined by the widely accepted GINA/NIH (Global Initiative For Asthma/National Institutes of Health) guideline-based treatment goals. Even with higher doses of inhaled corticosteroids most patients continue to require beta agonist bronchodilator therapy and current U.S. and international asthma treatment guideline recommend that patients with more than mild asthma be treated daily with both anti-inflammatory and long acting bronchodilator therapy. Many more severe asthmatics are actually treated with high dose inhaled corticosteroids as well as one or more additional anti-inflammatory drug daily in order to attain guideline directed levels of disease control and improved quality of life.

However, the deleterious side effects of these higher doses of inhaled corticosteroids given long-term often outweigh the clinical benefits for some patients. In addition, many studies have shown that patient adherence to long term daily treatment with two or more medications is very poor. In general, studies have shown highest compliance in chronic disease therapy can be achieved with once daily oral administration dosing regimens. For this reason, the search for better, orally administered, complementary “controller” treatments that can both spare asthma patient exposures to higher doses of Inhaled corticosteroids as well as reduce their number of daily medications and frequency of dosing has been widely advocated to provide better asthma control and prevent progression of the disease.

Role of Eosinophils in Asthma

The role of eosinophils in asthma is described in detail in Busse, W. W. et al., N. Engl. J. Med. 2001; 344-350, which disclosure is incorporated herein by reference. Inhaled antigens activate mast cells and Th2 cells in the airway, which in turn induce the production of mediators of inflammation such as histamine, leukotrienes and chemokines, including interleukin-4 and interleukin-5. Interleukin-5 in the bone marrow causes terminal differentiation of eosinophils. Circulating eosinophils enter the area of allergic inflammation and begin migrating to the lung by rolling, through interactions with selecting, and eventually adhering to the endothelium through the binding of integrins to members of the immunoglobulin superfamily of adhesion proteins: vascular-cell adhesion molecule 1 (VCAM-1) and intercellular adhesion molecule 1 (ICAM-1). As the eosinophils enter the matrix of the airway through the influence of various chemokines and cytokines (such as MCP-1, monocyte chemotactic protein, and MIP-1 (macrophage inflammatory protein), their survival is prolonged by interleukin-5 and granulocyte-macrophage colony-stimulating factor (GM-CSF). On activation, the eosinophil releases inflammatory mediators such as leukotrienes and granule proteins to injure airway tissues. In addition, eosinophils can generate granulocyte-macrophage colony-stimulating factor to prolong and potentiate their survival.

The presence of activated CD4 Th2 cells is also a hallmark feature of asthma in particular of chromic asthma. The persistence of Th2 cells may be the result of an increased recruitment and a prolonged survival in the airway tissue interstium (Cohn L, Elias J A, Chupp G L. Annual Review of Immunology. 2004. 22 (1): 789-815). As with eosinophils, Th2 cells enter the airways from the vascular through interaction of adhesion molecules with the vascular endothelium. Once in the tissue, these cells encounter antigen presenting cells, such as dendritic cells, where they proliferate. This costimulatory response as well as the resistance to apoptosis may be mediated by alpha4-VCAM-1 interactions.

Early and Late Phase Reactions to Allergens

In controlled inhaled allergen challenge experiments, sensitized asthmatic patients develop an early-phase allergic response (EAR) that begins within minutes of allergen exposure and most often resolves spontaneously after 30 to 60 minutes. This early-phase allergic response results primarily from the release of preformed pro-inflammatory mediators such as histamine as well as the de novo generation of leukotrienes C₄, D₄, and E₄ by bronchial mast cells. These mediators induce smooth muscle contraction, mucus secretion, and vasodilatation. Inflammatory mediators also induce microvascular leakage of plasma proteins, causing edematous swelling of the airway walls and a narrowing of the airway lumen.

This early-phase allergic response is usually followed by a second phase of airflow obstruction, termed the late-phase allergic response (LAR), which occurs 6 to 10 hours later. The late-phase allergic response develops as a result of cytokines and chemokines generated by resident cells of the lung (mast cells, macrophages, and epithelial cells) and recruited inflammatory cells (T lymphocytes and eosinophils). The T lymphocytes involved in this process are of the Th2 type and are found in a wide variety of hypersensitivity reactions including allergic rhinitis as well as asthma. Th2 cells produce interleukins, which have pronounced effects on inflammatory cells, particularly eosinophils. Circulating eosinophils migrate into the airway. Upon activation, eosinophils release inflammatory mediators such as leukotrienes, and granule proteins such as major basic protein which injure airway tissues. Features of the late-phase allergic response include bronchospasm, escalating inflammation, mucous hypersecretion and airway wall edema. Swelling of the airway wall also leads to a loss of elasticity, further contributing to chronic airflow limitation. An additional consequence of the late-phase allergic response is an increase in airway hyper-responsiveness, which reinforces and perpetuates the asthmatic response.

The Integrins

The integrins constitute a large class of heterodimeric, cell surface molecules consisting of α and β chains, each of which has a large extracellular domain and a short cytoplasmic tail. There are at least 14 different α chains and 8 β chains known, which combine in a restricted manner depending on cell type to give approximately 23 members of the integrin family, each of which binds specific peptide ligands. Integrins mediate a variety of cell functions including adhesion, migration, activation and survival. Lymphocytes and leukocytes with the exception of neutrophils constitutively express the integrin VLA-4 (α₄β₁, very late activating antigen-4, CD-49d/CD-29) and are capable of expressing the closely related integrin, α₄β₇.

The α₄β₁ and α₄β₇ integrins mediate cell-cell adhesion to the immunoglobulin superfamily member, vascular cell adhesion molecule-1 (VCAM-1), and cell-matrix adhesion to fibronectin. In addition, α₄β₇ also binds mucosal addressin cell adhesion molecule-1 (MadCAM-1). VCAM-1 regulates leukocyte migration from the blood into tissues. VCAM-1 expression is induced on endothelial cells during inflammatory responses such as that seen in asthma.

In asthma, there is increased expression of α₄β₁ and α₄β₇ integrins on all mononuclear leukocytes (including Th2 cells), eosinophils, basophils, and mast cells. The selective and increased expression of the α₄ integrins only on those cells involved in the inflammatory cascade in asthma would suggest that it is possible to target the underlying disease process without compromising normal host-defense responses.

In vivo studies with monoclonal antibodies (MoAbs) to the α₄ chain of α₄β₁ and α₄β₇ in several animal models of asthma demonstrate that α₄ integrins play a key role in eosinophil and T cell recruitment, activation, and survival leading to a significant reduction of airway inflammation. Furthermore, antibodies directed against VLA-4 block eosinophil accumulation, hyper-reactivity, and inflammation in mouse, rat and guinea pig models of allergic asthma. More recently the peptide VLA-4 antagonist, Bio1211, was shown to block late phase airway response as well as to attenuate carbacol induced airway hyper-responsiveness in a sheep model of allergic asthma. Lastly, VCAM-deficient mice show no signs of airway inflammation.

R411

R411 (N-(2-Chloro-6-methylbenzoyl)-4-[(2,6-dichlorobenzoyl)amino]-L-phenylalanine-2-(diethylamino)ethyl ester) is an ester pro-drug of the active moiety, N-(2-chloro-6-methylbenzoyl)-4-[(2,6-dichlorobenzoyl)amino]-L-phenylalanine. R411 has the following chemical structure:

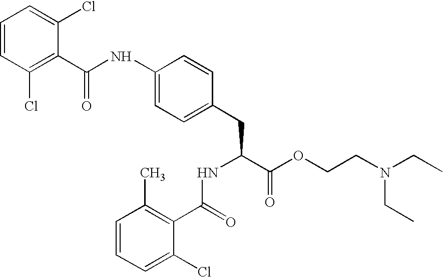

R411 inhibits the binding of α₄/β₁ to vascular cell adhesion molecule (VCAM-1) and α₄/β₇ to MadCAM-1 by binding to R411 is disclosed in U.S. Pat. No. 6,229,011, which disclosure is incorporated by reference herein.

R411 will only modulate immune responses mediated by α₄-integrins and, therefore in asthma, selectively target only those inflammatory cells involved in the pathogenesis of the disease: Th2 cells, eosinophils, and mast cells. The expression of α₄-integrins on these cells is increased in asthma mediating their recruitment, activation, retention, and survival in the airways. The alpha4 integrins appear not to be involved in cellular immunity and other humoral host defense responses. Therefore R411 would be expected to selectively target the inflammatory response in asthma without compromising normal host-defense.

R411 binds with high affinity and slow dissociation from the activated α₄ ligand. In contrast, in vitro binding affinity is lower and dissociation is more rapid when the receptor is not activated. While Bio1211 is specific for α₄/β₁ integrin, R411 is effective against both α₄/β₁ and α₄/β₇ integrins.

R411 can attenuate airway hyper-responsiveness; reduce edema; reduce smooth muscle hypertrophy/mucus gland hyperplasia; block trafficking of leukocytes to airways; increase peripheral blood lymphocytes and eosinophils; modulate Th2 cytokine production; block costimulatory signals for T cells and eosinophils; and inhibit eosinophil survival. In our experimental studies, R411 was observed to block the migration of key inflammatory cells from the blood into the lungs.

Many α-integrin inhibitors having various inhibitory selectivity patterns have been disclosed; see e.g.: U.S. Pat. Nos. 6,380,387; 6,388,084; 6,420,600; 6,423,728; 6,455,550; and 6,734,311.

Bambuterol

Bambuterol is an oral long-acting β₂-adrenergic agonist for the once-daily treatment of the symptoms of asthma. Bambuterol is dimethylcarbamic acid 5-[2-[(1,1-dimethylethyl)amino]-1-hydroxyethyl]-1,3-phenylene ester. Bambuterol is manufactured and sold by AstraZeneca as the hydrochloride salt (Bambuterol hydrochloride, Bambec®, oxeol) and has the following chemical structure:

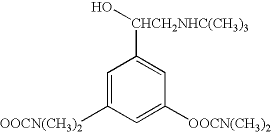

Bambuterol is disclosed in U.S. Pat. No. 4,419,364, which disclosure is incorporated by reference herein.

According to the AstraZeneca website, Bambuterol is a pro-drug that is slowly metabolized in the liver to the active form, terbutaline (5-[2-[(1,1-dimethylethyl)amino]-1-hydroxyethyl]-1,3-benzenediol)), thus providing a prolonged action. The metabolic conversion of bambuterol to terbutaline proceeds via intermediary metabolites by both oxidative and hydrolytic reactions. Bambuterol is accordingly a bis-dimethylcarbamate prodrug of terbutaline that can be used once-daily since it provides hydrolytic stability and specificity for hydrolysis by butyrylcholinesterases to liberate terbutaline. (Svennson L A, Tunek A. Drug Metabolism Rev 1988, 19 165-94). Bambuterol is a racemic product with a single chiral center. (−)-Bambuterol is responsible for the pharmacodynamic effects via generation of (−)-terbutaline and (+)-bambuterol generates the pharmacodynamic inactive (+)-terbutaline. Both (+) and (−)-bambuterol are equally active as plasma cholinesterase inhibitors.

Pharmacodynamic studies have shown that after oral administration of bambuterol to guinea pigs, a sustained protective effect was achieved against histamine induced bronchoconstriction. At equipotent doses, the duration of the relaxing activity was more prolonged than after administration of just terbutaline. Bambuterol, or the monocarbamate ester, did not exert any smooth muscle relaxing properties.

Bambuterol is supplied as tablets and as a liquid medicine for pediatric use. The recommended dosage for adults is 10-20 mg. Tablets include the following excipients: lactose monohydrate; maize starch; povidone; microcrystalline cellulose; magnesium stearate; and purified water. Bambuterol is not intended to treat acute asthma attacks. Bambuterol is approved for asthma treatment in 28 countries (Clinical Pharmacokinetics, 31 (4), 246-256, 1996).

### BRIEF DESCRIPTION OF THE FIGURES

FIG. 1 is a graph illustrating the additive effect of R411 on moderate dose inhaled corticosteroids in large airway flow rates as measured by FEV₁.

FIG. 2 is a graph illustrating the additive effect of R411 on moderate dose inhaled corticosteroids in large airway flow rates as measured by FEF25-75.

FIG. 3 is a graph illustrating the effect of R411 on small airway flow rates as measured by FEF25-75 when administered as monotherapy to asthmatic patients.

FIG. 4 is a bar graph showing that the oral administration of R411 attenuates airway inflammation in the atopic primate.

### SUMMARY OF THE INVENTION

The present invention provides a solid pharmaceutical dosage form for oral administration comprising a therapeutically active amount of bambuterol, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable salt thereof, and one or more pharmaceutically acceptable excipients.

The present invention also provides a method for treating asthma comprising administering to a subject, in need thereof, a solid pharmaceutical dosage form for oral administration comprising a therapeutically active amount of bambuterol, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

The present invention further provides a method for preparing a solid pharmaceutical dosage form for oral administration comprising admixing a therapeutically active amount of bambuterol, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

### DETAILED DESCRIPTION OF THE INVENTION

The present invention provides solid pharmaceutical dosage forms for oral administration comprising a therapeutically active amount of bambuterol, or a pharmaceutically acceptable salt thereof, a therapeutically effective amount of R411, or a pharmaceutically acceptable thereof, and one or more pharmaceutically acceptable excipients. In a preferred embodiment, the dosage form comprises a combination of bambuterol and R411 admixed together with pharmaceutical excipients or per-formulated individually and then mixed to form a unit dose containing a therapeutic amount of each compound. The first composition comprises a therapeutically active amount of bambuterol, or a pharmaceutically acceptable salt thereof, formulated with one or more pharmaceutically acceptable excipients. The second composition comprises a therapeutically effective amount of R411, or a pharmaceutically acceptable salt thereof, formulated with one or more pharmaceutically acceptable excipients. In a more preferred embodiment, the dosage form comprises two discrete regions. The first region comprises a therapeutically effective amount of bambuterol, or a pharmaceutically acceptable salt thereof. The second region comprises a therapeutically effective amount of R411, or a pharmaceutically acceptable salt thereof. These oral dosage forms are useful in the treatment or control of asthma and allergic rhinitis.

The pharmaceutical dosage forms of the present invention provide two compounds for treating asthma that operate by complementary mechanisms of action. Bambuterol is an oral β₂-adrenergic agonist useful to manage acute exacerbation of asthma as well as acute episodes of bronchospasm. Stimulation of β₂-adrenergic receptor sites results in the activation of adenyl cyclase, which increases the production of cyclic 3′,5-adenosine monophosphate resulting in bronchial smooth muscle relaxation and skeletal muscle alkalinizing agent stimulation, and the inhibition of the release of inflammatory mediators. R411 inhibits eosinophil and Th2 cell excitation and survival, and inhibits eosinophil migration from blood to pulmonary tissues. The combination of the two compounds in the pharmaceutical dosage forms therefore provides a therapeutic treatment that has the combined effect of inhibiting the release of inflammatory mediators and reducing eosinophil egress into pulmonary tissues thereby providing an early onset of bronchodilation as well as sustained anti-inflammatory effects. Hence administration of the pharmaceutical dosage forms of the present invention provides a means of intensifying asthma therapy while supporting good patient compliance.

Bambuterol is commercially available as the hydrochloride salt to provide the desired solubility and stability. R411 is also a weak base and is commonly used as the hydrochloride salt. The novel solid pharmaceutical dosage forms of the present invention take advantage of this structural similarity to provide formulations that have sufficient stability without compromising in vivo performance. Several formulations can be employed to provide these combination products. The most preferred method of manufacturing the combination product is by formulating the two therapeutically active components with pharmaceutically acceptable excipients and conventional manufacturing equipments. However, to further enhance the stability of the product, it has been discovered that it is preferable that the two active ingredients be first pre-formulated separately to obtain pharmaceutically acceptable stability and bioavailability characteristics for each ingredient. The two separately pre-formulated active ingredients are then combined in an appropriate solid dosage composition for oral administration. Other preferred solid dosage forms are those in which the separately pre-formulated ingredients are combined in a dosage form having separate discrete regions for the two pre-formulated ingredients such as by discrete layers, encapsulations, and the like. Examples of such dosage forms include, but are not limited to, a compressed tablet, a bilayer tablet, a sandwich tablet, a tablet having coated microbeads, or a film coated tablet.

As used herein, the following terms have the given meanings:

“Bambuterol” refers to bambuterol, pharmaceutically acceptable salts thereof, to racemic mixtures, and to pure enantiomers.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., H. Ansel et. al., Pharmaceutical Dosage Forms and Drug Delivery Systems (6^{th} Ed. 1995) at pp. 196 and 1456-1457.

“Prodrug” refers to compounds, which undergo biotransformation prior to exhibiting their pharmacological effects. The chemical modification of drugs to overcome pharmaceutical problems has also been termed “drug latentiation.” Drug latentiation is the chemical modification of a biologically active compound to form a new compound, which upon in vivo enzymatic attack will liberate the parent compound. The chemical alterations of the parent compound are such that the change in physicochemical properties will affect the absorption, distribution and enzymatic metabolism. The definition of drug latentiation has also been extended to include nonenzymatic regeneration of the parent compound. Regeneration takes place as a consequence of hydrolytic, dissociative, and other reactions not necessarily enzyme mediated. The terms prodrugs, latentiated drugs, and bio-reversible derivatives are used interchangeably. By inference, latentiation implies a time lag element or time component involved in regenerating the bioactive parent molecule in vivo. The term prodrug is general in that it includes latentiated drug derivatives as well as those substances, which are converted after administration to the actual substance, which combines with receptors. The term prodrug is a generic term for agents, which undergo biotransformation prior to exhibiting their pharmacological actions.

“R411” refers to N-(2-chloro-6-methylbenzoyl)-4-[(2,6-dichlorobenzoyl)amino]-L-phenylalanine-2-(diethylamino)ethyl ester, and pharmaceutically acceptable salts thereof.

“Therapeutically effective amount” means an amount of at least one compound of the invention, or a pharmaceutically acceptable salt thereof, which is effective to prevent, alleviate or ameliorate symptoms of disease or prolong the survival of the subject being treated. Determination of a therapeutically effective amount is within the skill in the art.

As set out above, one component in the solid pharmaceutical dosage form comprises a therapeutically effective amount of bambuterol, or a pharmaceutically acceptable salt thereof. Bambuterol is an oral β2-adrenergic agonist useful for the treatment of the symptoms of asthma. β2-adrenergic agonists are used to manage acute exacerbation of asthma as well as acute episodes of bronchospasm. Stimulation of β2-adrenergic receptor sites on the sympathetic nervous system results in activation of adenyl cyclase, which increases the production of cyclic 3′,5-adenosine monophosphate (cAMP). This increase in the production of cAMP results in bronchial smooth muscle relaxation and skeletal muscle alkalinizing agent stimulation, and inhibits the release of inflammatory mediators via stabilization of the mast cell membrane. This in turn slows progression of the inflammatory cascade. Bambuterol is metabolized in the liver to the pharmacologically active β2-adrenergic agonist terbutaline via intermediary metabolites by both oxidative and hydrolytic reactions.

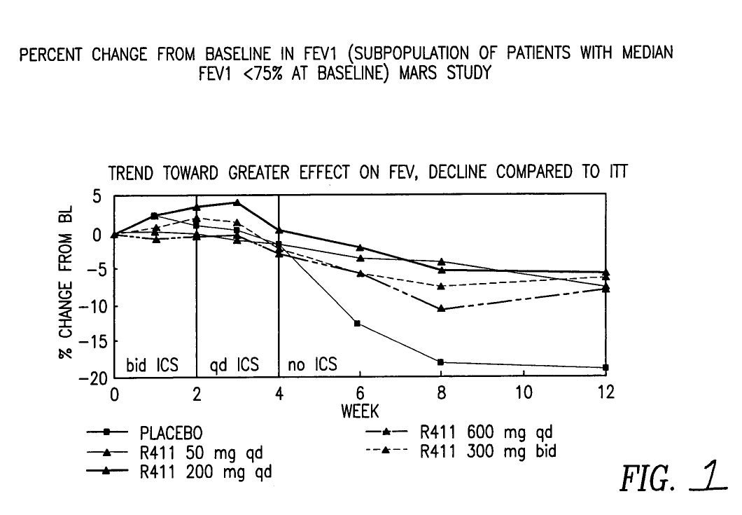

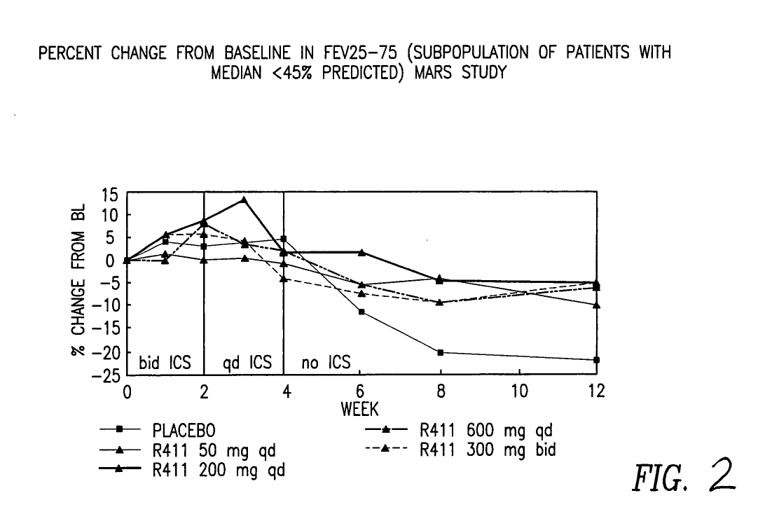

As set out above, a second component in the solid pharmaceutical dosage form comprises a therapeutically effective amount of R411 (N-(2-chloro-6-methylbenzoyl)-4-[(2,6-dichlorobenzoyl)amino]-L-phenylalanine-2-(diethylamino)ethyl ester), or a pharmaceutically acceptable salt thereof. In Phase II studies, R411 demonstrated an additive effect to moderate dose inhaled corticosteroids in large airway flow rates as measured by FEV₁ (FIG. 1) and small airway flow rates measured by FEF25-75 (FIG. 2) in a subpopulation of patients with not well controlled asthma. The MARS study illustrated in FIG. 2 was designed to evaluate the safety and efficacy of R411 over a 12 week treatment period in 350 persistent asthmatics being treated with a stable dose of low to medium inhaled corticosteroids and inhaled short acting β2-agonist. Patients were randomized to one of five cohorts: 50, 200, 600 mg once daily (QD) or 300 mg twice daily (BID) R411, or placebo (n=70/group). After a 2-week placebo run-in period and subsequent 2-week add-on period (R411 or placebo), morning inhaled corticosteroids were removed. Two weeks later, evening inhaled corticosteroids were removed, and patients remained in the treatment period for an additional 8 weeks. The primary endpoint in the study was the percentage change in FEV1 from baseline, and secondary endpoint included PEFR, asthma exacerbations, (2-agonist use, asthma control questionnaire, asthma symptom scores, nocturnal awakenings, FEF25-75 and rate of asthma treatment failures.

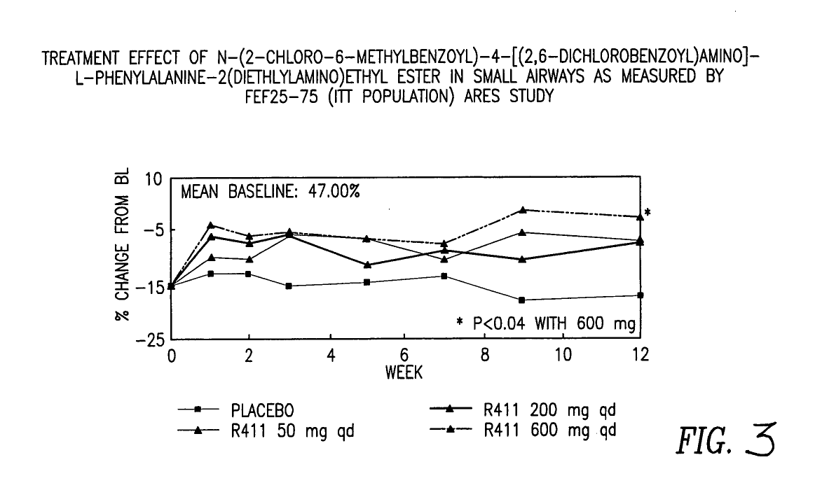

A significant effect on small airway flow rates as measured by FEF25-75 was seen with R411 even when administered as monotherapy to asthmatic patients (FIG. 3). The ARES study illustrated in FIG. 3 was designed to evaluate the safety and efficacy of monotherapy R411 over a 12 week treatment period in 480 mild/moderate asthmatics not treated with inhaled corticosteroids. Patients were randomized to one of four cohorts: 50, 200, 600 mg QD R411, or placebo (n=120/group). The primary endpoint in the study was change in FEV1 from baseline, and secondary endpoints included PEFR, asthma exacerbations, (2-agonist use, asthma control questionnaire, asthma symptom scores, and nocturnal awakenings. Small airway inflammation represents a clinically significant component of moderate to severe asthma that is not adequately controlled by currently available inhaled corticosteroids therapies. Therefore, R411 represents a novel opportunity to address an important unmet need in control of asthmatic airway inflammation.

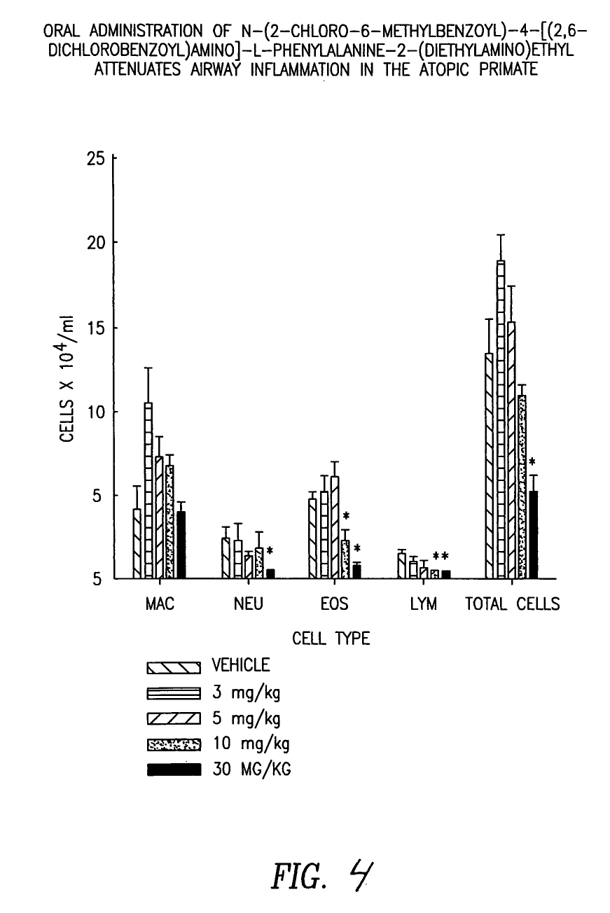

FIG. 4 is a bar graph showing that the oral administration of R411 attenuates airway inflammation in the atopic primate.

Improvement of Asthma Symptoms

In a clinical study of 26 patients, Bambec significantly increased PEF rates and significantly reduced the use of inhaled beta2-agonists, number of nocturnal awakenings, and nocturnal asthma symptom scores. More patients reported a significantly improved quality of sleep and overall state of health after Bambec treatment, than after placebo.
!! INCLUDEPICTURE “http://www.az-air.com/phy_prod/bilder/bambec/AR0112201.gif”\* MERGEFORMATINET ¶┌^{⊥} (Petrie G R, et al. Respir Med 1993; 87: 581-5.)

Bambec tablets were administered once daily in the evening for 4 weeks. PEF was measured daily immediately before the dose of Bambec was given. Bambec given once daily in the evening significantly improved evening PEF over the treatment period (Persson G, et al. Eur Respir J 1995; 8: 34-9).

R411 also has positive effects on symptoms of asthma. The ARES study evaluated the safety and efficacy of monotherapy with R411 over a 12-week treatment period in 479 mild/moderate asthmatics not treated with inhaled corticosteroids. Patients were randomized to one of four cohorts: 50, 200, 600 mg once daily R411, or placebo. Statistically significant improvements with R441 were achieved in reducing rescue albuterol use, decrease in daytime asthma and nocturnal symptom score. Improvement in Asthma Control Questionnaire Scores and Asthma Quality-of-Life were also observed when compared to placebo. Although the study was not powered to detect significant differences in asthma exacerbations, a 26% reduction was observed with the two highest doses of 200 and 600 mg. The results are set out in the Table below.

Secondary Efficacy Endpoints in the ITT Population when
R411 is Administered as Monotherapy (ARES Study)

| | Change from Baseline ITT Population | | |
|---|---|---|---|
| | (Median FEV1 74.75% at Baseline) Table | | |
| | Secondary Efficacy Endpoints in the ITT | | |
| | Population when R411 is Given as | | |
| | Monotherapy (ARES Study) | | |
| | Placebo | 200 mg | 600 mg |
| | (N = 117)/ | (N = 117)/ | (N = 119)/ |
| | [Mean BV] | [Mean BV]) | [Mean BV] |
| Rescue β 2-agonist use | 0.1/[2.98] | −0.36/[3.04] | −0.41*/[3.11] |
| (puffs/d) | | | |
| Nocturnal awakenings | 0.06/[0.54] | −0.15*/[0.61] | −0.12*/[0.58] |
| (scores) | | | |
| Morning asthma | −0.13 [1.61] | −0.34* [1.55] | −0.33* [1.57] |
| symptoms | | | |
| Asthma control | −0.07 [2.00] | −0.29 [2.08] | −0.23 [2.00] |
| Questionnaire (Total | | | |
| Score) | | | |
| % Asthma exacerbation | 32.50 | 25.6 | 26.10 |

*p < 0.05, before adjustments for multiple comparisons;
BV = baseline value for the group.

Solid Oral Dosage Forms Comprising Bambuterol and R411

In accordance with the present invention, solid pharmaceutical dosage forms for oral administration are provided comprising a therapeutically active amount of bambuterol (either as a racemic mixture or as a pure enantiomer), or a pharmaceutically acceptable salt thereof, a therapeutically effective amount of R411, or a pharmaceutically acceptable salt thereof, and one or more pharmaceutically acceptable excipients. In a preferred embodiment, the dosage form comprises a combination of two actives admixed with acceptable excipients in a once daily oral dosage form. These once daily oral dosage forms are useful in the treatment or control of asthma.

Without intending to limit the invention to any particular theory, the pharmaceutical dosage forms of the present invention are believed to provide an improved efficacy profile in the treatment of asthma by virtue of their complementary mechanisms of action. Bambuterol is an oral β₂-adrenergic agonist useful to manage acute exacerbation of asthma as well as acute episodes of bronchospasm. Stimulation of β₂-adrenergic receptor sites results in the activation of adenyl cyclase, which increases the production of cyclic 3′,5-adenosine monophosphate resulting in bronchial smooth muscle relaxation and skeletal muscle alkalinizing agent stimulation, and the inhibition of the release of inflammatory mediators. The specific mechanism of action of R411 suggests that it's greatest effect will be on the late-phase allergic response in animal and human challenge studies characterized by its effect on eosinophils. R411 inhibits eosinophil excitation and survival, inhibits eosinophil migration from blood to pulmonary tissues, and may promote apoptosis of tissue eosinophils though integrin blockade. Administration of a solid oral dosage form containing both bambuterol and R411 would therefore provide a therapeutic treatment having the combined effects of a β2-adrenergic agonist, which stimulates β2-adrenergic receptor sites and inhibits the release of inflammatory mediators and reducing eosinophil egress into pulmonary tissues. Administration of the dosage form containing both compounds of the present invention provides an improved lung function than that achieved by administration of either drug alone by virtue of their complementary modes of action.

The therapeutically effective amount or dosage of bambuterol and R411 according to this invention can vary within wide limits and may be determined in a manner known in the art. Such dosage will be adjusted to the individual requirements in each particular case including the specific compound(s) being administered, the condition being treated, as well as the patient being treated. In general, in the case of oral administration of bambuterol, or pharmaceutically acceptable salts thereof, to adult humans weighing approximately 70 Kg, bambuterol will be present in a daily dosage ranging from about 5 mg to about 30 mg, preferably from about 10 mg to about 20 mg. In general, in the case of oral administration of R411, or pharmaceutically acceptable salts thereof, to adult humans weighing approximately 70 Kg, R411 will be present in a daily dosage ranging from about 50 mg to about 400 mg, more preferably from about 50 mg to about 200 mg.

As set out above, because of the structural similarity of bambuterol and R411, it is possible to prepare a fixed dose combination product by admixing the two actives with pharmaceutically acceptable excipients. Alternatively, the two active ingredients be first pre-formulated separately to obtain pharmaceutically acceptable stability and bioavailability characteristics for each ingredient and then combined in an appropriate solid dosage composition for oral administration. Examples of such dosage forms include, but are not limited to, a compressed tablet, a bilayer tablet, a sandwich tablet, a tablet having coated microbeads or film-coated tablets. These tablets could also designed to have fast disintegration in the oral cavity for administration to patients with swallowing difficulty such as children and the elderly.

In general, bilayer tablets may be formulated by utilizing twin hopper compression machines. The granulates of each compound may be prepared individually using pharmaceutically acceptable excipients such as lactose, sucrose, microcrystalline cellulose, stearic acid, hydroxypropylmethylcellulose, low substituted hydroxyprolylcellulose, polyvinylpyrrolidone, maize starch crospovidone, croscarmelose sodium, sodium starch glycolate, microcrystalline cellulose, starch, dicalcium phosphate, mannitol, sorbitol, silicified microcrystalline cellulose, talc, colloidal silica, stearic acid, or magnesium stearate. The individual granulates can then be compressed together into one unit.

In general, sandwich tablets (or tablets inside tablets) can be prepared by sandwiching a tablet of bambuterol unit into the granulates of R411 using twin hopper compression machines. The tablet of bambuterol is prepared by using standard excipients described above and the granulates of R411 are prepared by conventional granulation techniques using pharmaceutically acceptable excipients.

In general, tablets having coated microbeads can be prepared by formulating one of the components, such as bambuterol, using either granulation or granulation followed by extrusion-merumerization techniques and coating the component with pharmaceutically acceptable polymers such as hypromellose, ethylcellulose, hydroxypropylcellulose, polyvinylalcohol, and/or aminomethylmethacrylate Eudagit E100 or Eudragit EPO or Eudragit L100 (Rohm America, NJ) in fluid bed or coating pans in such a proportion that coating provides enough barrier to separate the two active components but does not affect the dissolution behavior of the coated product. The coated microbeads of bambuterol can then be mixed with R411 granulates prepared using conventional methods. These mixed granulations can be used to prepare tablets, capsules, or suspensions, or can be dispersed in an oily matrix. Separating the granulation process and further coating of those granulates help provide the barrier required to keep the two components separate while not affecting the dissolution behavior thus assuring the desired pharmacokinetic exposures. This approach can be used for taste masking each active separately and then mixing together with pharmaceutically acceptable excipients such as Pharmaburst® (SPI Pharma, DE), crospovidone, FM1000 (J. M. Huber Corp, MD), microcrystalline cellulose , xylitol, mannitol, sugar, lactitol, maltitol, sorbitol, sucralose, aspartame, sodium saccharin, maltodextrin, fructose, dextrose, Avicel CE (FMC Biopolymer, PA), colloidal silicone dioxide, etc. to yield an orally disintegrating product.

In general, film-coated tablets can be prepared by incorporating bambuterol in a film-coating layer. Tablets of R411 are prepared by conventional manufacturing processes such as granulation, milling, blending, lubricating, and compressing. The required dose of bambuterol is dissolved or dispersed in a coating dispersion usually consisting of film forming agents such as hypromellose (hydroxypropyl methylcellulose), polyvinyl alcohol, starch or ethylcellulose along with a gliding agent such as talc, colorant and plasticizer (triacetin, dibutylsebacate, polyethylene glycol) dispersed in water. The required amount of bambuterol film coating is then applied over the R411 kernel tablet either in a pan coater or fluidbed coater to deposit the specific amount of bambuterol onto the R411 kernels.

The process of granulation consists of granulation with water or an appropriate solvent in a low or high shear granulator, fluid bed dryer, dry granulation with roller compaction or slugging or melt granulation using polyethylene glycols, phospholipids, poloxamers, monoglycerides, diglycerides and triglycerides, fatty acids, polyglycolized ester such as Gelucires, Vitamin E TPGS or by melt extrusion using thermosetting polymers such as polyvinylpyrrolidone, copolyvidone, poloxamers, polyethylene glycol, ethyl cellulose, stearic acid, glyceryl monostearate, glyceryl behenate, and/or sucrose diesters. In order to manufacture the oral suspension, transdermal patches, these granulates in the desired proportion are dispersed in pharmaceutical bases consisting of excipients such as polyethylene glycols, surfactants Cremophor EL, Cremophor RH40, Solutol HS15, Gelucires 44/14, 50/15, 39/01, 33/01, (BASF Pharma Solutions), Gelucires 44/14, 50/15, 39/01, 33/01 (Gattefossse Corp, NJ), Capmul MCM, Capmul PG 8(Abitec, OH), polysorbates, spans, sodium dodecyl sulfate can be added to further improve the absorption process.

In another embodiment, the present invention provides a method for treating asthma comprising administering to a subject, in need thereof, a solid pharmaceutical dosage form for oral administration comprising a therapeutically active amount of bambuterol, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients. In yet another embodiment, the present invention provides a method for preparing a solid pharmaceutical dosage form for oral administration comprising admixing a therapeutically active amount of bambuterol, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

The pharmaceutical dosage forms of the present invention can be prepared according to the examples set out below. Excipients must be selected to assure the stability of the products since it has been found that both bambuterol and R411 are susceptible to hydrolytic degradation to differing extents. Furthermore, both compounds are weak bases and the solubility and stability of the compounds are sensitive to counter-ions. In order to assure the stability of the product and to achieve desirable solubility of the dosage form, the choice of excipient is critical. Including an acidulating agent such as fumaric acid, adipic acid, succinic acid, tartaric acid, citric acid, lactic acid, maleic acid, etc., in the formulation, as well as eliminating soluble cations, is important to assure the solubility and stability of the product. The examples are presented for purposes of demonstrating, but not limiting, the preparation of the compounds and compositions of this invention.

### EXAMPLES

In accordance with the present invention, the following examples are provided to illustrate the solid pharmaceutical dosage forms of the present invention.

### Example 1

Conventional Compressed Tablets

Compressed tablets are formulated using conventional processing equipment. A typical composition is shown below illustrating the composition of a tablet manufactured using conventional processes such as granulation, compression and film-coating but with the careful selection of excipients.

| | |
|---|---|
| bambuterol | 5% |
| R411 | 50% |
| Povidone K30 | 4% |
| crospovidone | 4% |
| fumaric acid | 5% |
| lactose monohydrate | 23% |
| microcrystalline cellulose | 5% |
| talc | 3% |
| magnesium stearate | 1% |

A tablet weighing 200 mg of the above composition will yield 10 mg bambuterol and 100 mg of R411. A tablet weighing 400 mg will provide 20 mg bambuterol and 200 mg of R411.

### Example 2

Coated Microbeads of Bambuterol Mixed with Granulates of R411

Tablets having coated microbeads can be prepared by formulating one of the components, such as bambuterol, using either granulation or granulation followed by extrusion-merumerization techniques and coating the component with pharmaceutically acceptable polymers in fluid bed or coating pans in such a proportion that coating provides enough barrier to separate the two active components but does not affect the dissolution behavior of the coated product. Alternatively the beads can be prepared either as a minitablet prepared by compressing the granules followed by coating or by coating the drug dispersed in polymers onto sugar spheres in fluid bed. The coated microbeads of bambuterol can then be mixed with R411 granulates prepared using conventional methods.

A typical composition of a R411 tablet having coated microbeads of bambuterol is set out below.

a granulate comprising in percentages by weight of the tablet;

| | |
|---|---|
| R411 | 50% |
| povidone K30 | 4% |
| crospovidone | 4% |
| lactose hydrous | 26% |
| microcrystalline cellulose | 10% |
| talc | 5% |
| magnesium stearate | 1% |

coated microbeads in percentages by weight of the coated microbeads;

| | |
|---|---|
| bambuterol | 10% |
| microcrystalline cellulose | 78% |
| hypromellose | 5% |
| crospovidone | 2% |
| opadry complete coating system | 5% |

In this example, 100 mg of bambuterol microbeads and 400 mg of R411 granulates are compressed into tablets or filled into capsules to provide a fixed combination containing 10 mg of bambuterol and 200 mg of R411.

A typical manufacturing procedure for tablets having coated microbeads is set out below.

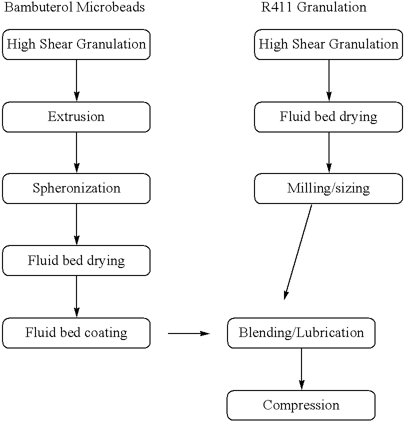

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

We claim:

1. A solid pharmaceutical dosage form for oral administration comprising a therapeutically active amount of bambuterol, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable salt thereof, and one or more pharmaceutically acceptable excipients.

2. The dosage form according to claim 1, wherein bambuterol is present in an amount from about 5 mg to about 30 mg.

3. The dosage form according to claim 1, wherein N-(2-chloro-6-methylbenzoyl)-4-[(2,6-dichlorobenzoyl)amino]-L-phenylalanine-2-(diethylamino)ethyl ester is present in an amount from about 50 mg to about 400 mg.

4. The dosage form according to claim 1, wherein the dosage form is selected from the group consisting of a compressed tablet, a bilayer tablet, a sandwich tablet, a tablet having coated microbeads, and a film coated tablet.

5. The dosage form according to claim 4, wherein the compressed tablet comprises:

| | |
|---|---|
| bambuterol | 5% |
| R411 | 50% |
| hydroxypropyl cellulose | 4% |
| crospovidone | 4% |
| fumaric acid | 5% |
| lactose hydrous | 23% |
| microcrystalline cellulose | 5% |
| talc | 3% |
| magnesium stearate | 1% |

6. The dosage form according to claim 4, wherein the tablet having coated microbeads comprises:
(a) a tablet comprising N-(2-chloro-6-methylbenzoyl)-4-[(2,6-dichlorobenzoyl)amino]-L-phenylalanine-2-(diethylamino)ethyl ester present in an amount from about 50 mg to about 400 mg; and
(b) coated microbeads dispersed throughout the tablet comprising bambuterol present in an amount from about 5 mg to about 30 mg.

7. The dosage form according to claim 5, wherein the tablet having coated microbeads comprises:
(a) a tablet comprising bambuterol present in an amount from about 5 mg to about 30 mg; and
(b) coated microbeads dispersed throughout the tablet comprising N-(2-chloro-6-methylbenzoyl)-4-[(2,6-dichlorobenzoyl)amino]-L-phenylalanine-2-(diethylamino)ethyl ester present in an amount from about 50 mg to about 400 mg.

8. The dosage form according to claim 5, wherein the tablet having coated microbeads comprises:
(a) a granulate comprising in percentages by weight of the tablet;

| | |
|---|---|
| R411 | 50% |
| povidone K30 | 4% |
| crospovidone | 4% |
| lactose hydrous | 26% |
| microcrystalline cellulose | 10% |
| talc | 5% |
| magnesium stearate | 1% |

(b) coated microbeads in percentages by weight of the coated microbeads;

| | |
|---|---|
| bambuterol | 10% |
| microcrystalline cellulose | 78% |
| hypromellose | 5% |
| crospovidone | 2% |
| opadry complete coating system | 5% |

9. The dosage form according to claim 1, wherein the pharmaceutically acceptable excipient is an acidulating agent.

10. A method for treating asthma comprising administering to a subject, in need thereof, a solid pharmaceutical dosage form for oral administration comprising a therapeutically active amount of bambuterol, or a pharmaceutically acceptable salt thereof, a therapeutically effective amount of N-(2-chloro-6-methylbenzoyl)-4-[(2,6-dichlorobenzoyl)amino]-L-phenylalanine-2-(diethylamino)ethyl ester, or a pharmaceutically acceptable thereof, and one or more pharmaceutically acceptable excipients.

11. The method according to claim 10, wherein bambuterol is present in an amount from about 5 mg to about 30 mg and N-(2-chloro-6-methylbenzoyl)-4-[(2,6-dichlorobenzoyl)amino]-L-phenylalanine-2-(diethylamino)ethyl ester is present in an amount from about 50 mg to about 400 mg.

12. The method according to claim 10, wherein the dosage form is selected from the group consisting of a compressed tablet, a bilayer tablet, a sandwich tablet, a tablet having coated microbeads, and a film coated tablet.
